(19) United States
(12) Patent Application Publication
(10) Pub. No.: US 2026/0295123 A1
(43) Pub. Date: Oct. 1, 2026

(54) DEVICES AND METHODS FOR PREPARING AND CONDITIONING BIOLOGICAL ASSEMBLIES FOR MINIMALLY INVASIVE DELIVERY FOR TISSUE ENGINEERING, CELL THERAPIES AND OTHER THERAPEUTIC APPLICATIONS

(71) Applicant: VOLUMINA MEDICAL SA, Lausanne (CH)

(72) Inventors: Amélie BÉDUER, Pully (CH); Thomas BRASCHLER, Chavannes-Pres-Renens (CH)

(21) Appl. No.: 19/480,350

(22) PCT Filed: Apr. 19, 2024
(86) PCT No.: PCT/CH2024/050022

§ 371 Date: Oct. 30, 2025

(30) Foreign Application Priority Data
May 3, 2023 (WO) PCT/CH2023/050011

(51) Int. Cl. A61L 27/36; A61J 1/20

(52) U.S. Cl. CPC A61L 27/3691; A61J 1/2089; A61L 27/3604; A61L 27/3687; A61L 2430/34

(57) ABSTRACT

The sterile container, preferably in form of a syringe, has an oxygen and/or carbon dioxide permeability of at least 0.1 mL/Pa/m²/day and is provided with at least one fluidic port permitting sterile connection to other containers and/or tubes, containing a porous, biocompatible polymer-based biomaterial with at least 30% porosity, preferentially at least 50% porosity, and most preferentially at least 80% porosity. This has the advantage of enabling intraoperative conditioning, circumventing the need for cell culture facilities, while still achieving targeted conditioning.

### BACKGROUND OF THE INVENTION

### 1. Field of the Invention

The invention relates to a container according to the preamble of claim 1, to an assembly according to the preamble of claim 18 and to a method for obtaining a conditioned biological assembly according to the preamble of claim 23.

### 2. Description of the Related Art

Numerous injectable or prosthetic devices exist that permit to restore or augment tissues in the human body. A very wide variety of such devices exist, for instance range from orthopedic and dental hard prosthetics, to firm implants such as silicone breast implants, to soft injectable dermal fillers.

These devices are biocompatible and are typically designed for restoring a given mechanical function, strength or physical appearance. They inevitably trigger a tissue reaction once implanted, but more often than not, regeneration is incomplete or utterly absent.

Attempts are increasingly being made at alleviating the shortcoming of incomplete regeneration. One approach consists in more refined, biomimetic implants; a prime example of this are various decellularized matrices (for instance, US 2012/0264190, WO2017114902A1 and others), other approaches are the use of guiding fibers in a hydrogel matrix or porous (U.S. Pat. No. 10,463,768), materials (U.S. Pat. No. 10,463,768B2, WO2019/068207, WO2012048165A2). While progress is being made, these approaches often fall short of full tissue regeneration.

This contrasts with a main aim of regenerative medicine, which is the creation of functional tissues. There are numerous approaches in regenerative medicine, which can be broadly grouped into techniques involving in-vitro preparation and subsequent grafting of living compositions on the on hand, and modulation or induction of in-vivo tissue regeneration on the other.

To achieve the aim to create new tissues in-vitro, cells are typically cultured under specific conditions in cell culture laboratories, and used alone or combined with biomaterials in order to create the new tissues. There are many examples of this strategy, for example U.S. Pat. No. 7,815,926B2 (cartilage from chondrocytes on demineralized bone) and US20140377863A1 (synthetic airways by culturing an airway scaffold with various cell suspensions), WO2021110908A1 (device for compacting microcarrier cultures for in-vivo delivery) and KR101373518B1 (encapsulation of pancreatic islets for xenotransplantation) and WO 2019/034550 A1 (genetically modified keratinocytes on fibrin flaps).

Some systems are adapted for the use with autologous materials, for example US2008/0286241 describes the in-vitro differentiation of small adipocytes from pre-adipocytes along with their subsequent delivery along with biodegradable scaffolds such as to obtain subsequent volume growth in-vivo. US20160113863A1 describes a mixture of ex-vivo expanded and native lipoextract. Ex-vivo tissue culture, isolation, and incubation steps are associated with stringent regulatory requirements in terms of contamination-free, traceable work environment (GMP). These facilities are not always available, and certainly add substantial economic cost even if they are. In any case, ex-vivo tissue generation remains heavily dependent on laboratory facilities.

The second important approach of regenerative medicine is the favorable modulation of endogeneous tissue processes in the host, such as vascularization, innervation, Immune response, lymphatic vascularization, and generally endogeneous tissue differentiation, proliferation, involution or other adaptation. Pharmacological molecules, but also very importantly growth factors, hormones, metabolites, cytokines and other mediators which are also secreted by living cells (the secretome) are effective for this aim.

Cell culture for ex-vivo tissue generation and optimal usage of the secretome are not completely in line. By culturing cells in flasks or bioreactors as currently performed in the field, the molecules secreted by the cultured cells, organoids, or fragments or tissue constructs are diluted in large volumes of cell culture medium and largely lost upon passaging, medium change or cell isolation before transplantation. The loss of a large part of the secretome before implantation is unfortunate because the molecules that have already secreted by cells would very often be extremely useful to achieve targeted cell and tissue behaviours in vivo. While cells are anticipated to continue to produce part of the secretome upon transplantation, conditions in-vivo are typically less controlled and less favorable for the generation of a particular secretome.

In part to achieve specific differentiation, but also in part to overcome the lack of suitable growth factors or other secretome components, synthetic growth factors or pharmacological agents can be added individually to compositions. For example, U.S. Pat. No. 10,688,223B2 describes cleavable attachment of growth factors and other biologically active molecules to porous scaffolds; US 2011/0307073 A1 describes a porous bone regeneration scaffold that can be infused with bioactive components; U.S. Pat. No. 9,610,328B2 describes an alginate hydrogel containing both VEGF165 and IGF-1 along with myogenic cells for muscle regeneration. The addition of exogenous growth factors however adds experimental and regulatory complexity, and may also induce safety risks in terms of immunological reaction to the products, to endotoxin contained therein, or by exaggerated or aberrant individual reaction.

Another method for making use of molecules secreted by cells in culture is to constitute what is known as conditioned medium. Conditioned media are complex mixtures of molecules secreted by cells, organoids, fragments or tissues during culture, generally containing both soluble factors and larger components such as exosomes, lipid and lipo-protein particles and sometimes extracellular matrix fragments. An example of a conditioned medium preparation is described in US20130217069A1, where conditioned medium is produced by embryonic or fetal fibroblasts, preferentially under hypoxic conditions for enrichment of the conditioned medium by keratinocyte growth factor. Likewise, EP 2201954A1 describes a pharmaceutical preparation from the supernatant of cultured peripheral blood mononuclear cells, optionally apoptotic or stressed, for modulating inflammation and tissue regeneration in the infarcted heart, for administration by injection.

For the production of conditioned medium, it is often necessary to proceed to concentration steps. In flask- or dish-based classical cell culture, this is in large part necessary to compensate for the dilution of the secreted molecules in the necessary large media volumes. However, even in denser culture systems such as bioreactor Systems or gas exchange systems (e.g. US2014/0273077A1 describing gas permeant cell culture systems), there are intrinsic limitations in the growth factor concentrations that can be achieved in culture. In most unaltered cells, negative feedback mechanisms operate to limit the effective concentration of growth factors and other signalling molecules even under favorable conditioning. Receptor-ligand complexes are also generally internalized by cells, often leading to ligand degradation (Cullen and Steinberg, Nat Rev Mol Cell Biol. 2018 November; 19 (11): 679-696. Doi: 10.1038/s41580-018-0053-7). Additionally, specific negative feedback loops exist: For example, high VEGF-A levels induce expression of soluble VEGFR-1, which neutralizes VEGF-A (Saito T et al. FEBS Lett. 2013 Jul. 11; 587 (14): 2179-85. Doi: 10.1016/j.febslet.2013.05.038). Both degradation after internalization and self-regulatory inhibition limit the effective growth factor concentrations that can be achieved in culture, regardless of the cell density. For this reason, and to remove waste products, conditioned media are often concentrated, for instance through the use of size-selective membranes to retain valuable factors based on their higher molecular weight.

Much like ex-vivo cell culture steps, the steps of medium collection and concentration add substantial regulatory and practical complexity. Avoidance of contamination, the presence of additional contact materials, and also additional immunogens due to the use of allogenic or xenogenic components in culture media or inherent in the cells used are some of the problems.

Summarizing, there is a need for technical solutions addressing efficiently both the aspect of in-vitro generation of new tissue precursors, and facile transfer of secreted molecules from the culture or conditioning step to the implantation step.

To be widely applicable in a clinical setting, such a system should be able to make use direct use of minimally altered surgical material as a starting point for tissue regeneration. Autologous tissue extracts are indeed readily available during surgical interventions; this is particularly true for lipoaspirate, but also blood and plasma, and in some circumstances other cell and tissue types such as fibroblast, keratinocytes of the skin, bladder, cornea, oral cavity or other mucous membranes, neurons or neural stem cells, peripheral bone marrow cells, immune cells and others, Lipoaspirates, plasma, or enriched fractions thereof such as stem-cell enriched lipoaspirate or platelet enriched plasma have been used to increase the regeneration capacity of biomaterial injectables such as hyaluronic acid and others. These materials can be used intra-operatively without specific need for GMP cell culture facility, avoiding many of the challenges and risks associated with ex-vivo cell culture. In this context, a number of devices further address intra-operative preparation of living implantables. U.S. Pat. No. 8,119,121B2 claims an autologous adipose implant enriched in stem cells, and describes a device useful for the closed-circuit production thereof. US20200164121A1 describes a device for the preparation of platelet-rich plasma. U.S. Pat. No. 8,221,394 B2 describes a device for liposuction allowing to soften or liquefy the material aspirated during liposuction by admixing a liquid. Biomaterials are also increasingly used along with such intraoperatively obtained materials. Indeed, many biomaterials for use in volume regeneration or augmentation can be used in conjunction with autologous tissues, and namely lipoaspirate. Some examples are silk particles (U.S. Pat. No. 9,931,434B2) and porous elastic microparticles (WO2017029633).

The combination of biomaterials and autologous tissues, or tissue extracts such as lipoaspirates is certainly beneficial in terms of combining immediate reconstruction with enhanced long-term regeneration. There is however room for fundamental improvement: These techniques generally neglect the importance of induction and accumulation of a target secretome. This is precisely because conditioning of cells contained in the extract remains limited or impossible with present techniques. This is a severe limitation, as the exact conditions under which biological extracts, cells, or assemblies of biological material and biomaterials are handled up to the point of injection or re-injection into a patient are of utmost importance for the biological and therapeutic outcome. The state of the cells themselves (i.e. genetic, epigenetic, transcriptional, proteomic, post-translational and signalling state) achieved through conditioning is clearly a key factor, as this at least partly determines the function of the cells upon transplantation. In addition, as discussed above, there is also a secretome accumulating during conditioning consisting of growth factors, hormones, metabolites and other signaling molecules secreted by cells, as well as larger aggregates such as exosomes, lipid vesicles, and possibly cell debris, extracellular matrix fragments, mitochondria, ribosomes, enzymes, ribozymes, genetic material, RNA another cell-released material. This accumulated secretome is a second factor of extreme importance for the therapeutical outcome. For instance, it has been shown that the secretome of mesenchymal stem cells, as released from alginate microcapsules, has striking immunomodulatory properties in-vivo (Mohammadi M et al, Adv Healthc Mater. 2020 June; 9 (12): e1901874. doi: 10.1002/adhm.201901874). The control over release kinetics is an additional feature that can be controlled through the use of this invention. Indeed, when using cell, organoid, or tissue suspensions, the release of factors already contained in the preparation will be rapid and insufficiently controlled in vivo. Typically, free growth factors injected with along with cells will cause an initial burst activity, to be rapidly lost as the molecules diffuse into the surrounding tissue and are neutralized, absorbed, or eliminated through lymphatic flow. This outlines a need for a secretome transfer system that not only induces and protects a suitable secretome throughout conditioning and transplantation, but also tailors in-vivo release.

Summarizing, there is at present a lack of devices, implantable biomaterials and methods that enable conditioning of cells and biological extracts, and provide for subsequent direct and controlled transfer and release of the secretome accumulated during condition.

It is an aim of this invention to provide devices, kits and methods for conditioning of cells, for example in biological extracts, in the presence of biomaterials. It is further aim of this invention to provide means to preserve or enhance the secretome, or specific parts of it, present at harvest of the biological extract or as generated during subsequent conditioning. And it is thirdly an aim of this invention to provide bioimplants with conditioned cells along with their secretome, for implantation and release in-vivo. The system is provided as closed but gas-permeable, optionally to be used at the bedside, permitting to avoid extended ex-vivo cell culture and associated risks and hurdles.

It is the object of this invention to describe a kit of devices, and methods of using them, to collect, prepare and assemble a biological assembly, or bioimplant, and deliver it in the target tissue or organ to elicit or augment favorable biological responses following implantation or re-implantation.

The aim of this invention is to provide methods and kits of devices to enable minimally invasive delivery/injection of biological assemblies (cells, biological extracts, biomaterials, and mixtures and derivatives thereof) with precise conditioning, in the context of tissue engineering, cell therapy, gene therapy, wound healing and others.

A preferred method of creating the bioimplant according to the invention applies conditioning conditions within the very syringe or container that will be used for collecting the biological extract and/or for the delivery thereof, as opposed to separation methods or culture methods in other recipients according to prior art. This has the advantage of enabling intraoperative conditioning, circumventing the need for cell culture facilities, while still achieving targeted conditioning. It has the further advantage to be implementable at the bedside of the patient, as a “closed process” without having to worry about contamination risks as opposed to conditioning performed traditionally in GMP cell culture labs and in cell culture flasks. Control of the conditioning environment in this case is ensured through a combination of biomaterial, gas permeable conditioning device and optionally cell culture media components. The presence of biomaterial during the condition step permits entrapment, or affinity-based retention of molecules secreted by the cells of the biological extract. Compared to prior art as disclosed in US 2014/0273077 A1 (gas permeant culture system), the invention provides the functionality of injectability and secretome entrapment. Compared to prior art regarding various assemblies of cells, hydrogels and growth factors (e.g. U.S. Pat. No. 9,610,328B2), the system avoids external growth factors and cells. Compared to prior art regarding conditioned media, it avoids external cell culture and processing steps.

### BRIEF SUMMARY OF THE INVENTION

The Invention solves the posed problem of conditioning, internal secretome accumulation and transfer of both conditioned biological extract and at least parts of interest of the secretome during implantation with a device comprising the features of claim 1, with an assembly enabling to condition biological extracts from a patient or from other sources comprising the features of claim 18, a method for obtaining a conditioned biological assembly comprising the features of claim 23.

In a preferred embodiment, the device consists in a sterile and closed, gas permeant container, which in a most preferred embodiment is a gas permeant syringe or container, containing an injectable, sterile and expansible biomaterial that in a most preferred embodiment is porous with interconnected pores, which is in a state of hydration or compression that provides it with the capacity to absorb at least 10%, preferentially at least 20%, more preferentially more than 50%, and most preferentially at least 100% of its own weight of additional liquid. The container, while being permeant to gases such as oxygen and optionally further gases such as carbon dioxide, nitrogen oxides, dinitrogen oxide, carbon monoxide, hydrogen sulfide, dimethylsulfide, hydrogen cyanide and others, and is impermeant to microorganisms. In one embodiment, this is obtained through a porosity in the range of 2 nanometers to 0.22 micrometers of the lining of the container, in another, it is obtained through diffusivity or enhanced diffusivity of gases through the lining. The device further has at least one port permitting sterile connection to a source of biological extract or cell suspension, and another, possibly identical, port for delivery of the condition implant for minimally invasive or open implantation into or onto a patient. Delivery may happen through simple extrusion of the bioimplant, or through a needle, cannula, dedicated applicators, endoscopic tubes and other means known in the art. The device or device port may further be equipped with structural features for facilitating static mixing, which are screw-like groves or screw-like traversing features generally known in the art; flexible bags or tubes can also be used for mechanical mixing. Optionally, the device can have further ports, identical or not to the main port, for medium addition or withdrawal, as well as addition or withdrawal of biomaterial or further living components such as external cell suspensions. Optionally, the biomaterial is stored in a separate container and only added to the gas-permeant container before use or after addition of the biological extract.

### A BRIEF DESCRIPTION OF THE DRAWINGS

FIG. 1 illustrates schematically an embodiment of the invention with the syringe or container used to condition the biological assembly contained therein, in an environment and within a defined incubation protocol. Accessories used for the harvest and injection procedures are also shown.

FIG. 2 illustrates the use case no. 1 schematically showing the collection, mixing, conditioning and delivery as steps of the method according to the invention.

FIG. 3 illustrates the use case no. 2 directed to a single multifunctional use of the collection syringe or container

FIG. 4 illustrates the use case no. 3 directed to the fluid replacement

FIG. 5 illustrates a schematic, longitudinal section through the gas-permeant syringe of container according to the invention; and

FIG. 6 shows a 3D view of the gas permeant barrel of the syringe or container according to FIG. 5.

### DEFINITIONS

The following definitions provide an outline for the relevant concepts for the devices and methods of use of this invention.

### Environment

The environment provides the physical and chemical conditions under which the conditioning is executed. The environment may provide control over conditioning gases, control for humidity and temperature and other functions, such as mechanical agitation.

### Biological Assembly or Bioimplant

A biological assembly, or bioimplant, is produced by mixing biomaterial, medium and biological extract, and modified throughout the processing taught by this invention. Through the use of the teachings of this invention, the biological assembly is then destined to be injected or implanted into a patient or an animal. The biological assembly typically consists of the biomaterial, the biological extract, and conditioning, storage or delivery fluid, but it may also consist of selected parts of the contents of the conditioning syringe or container after conditioning. This can be biomaterial having undergone contact with the biological extract alone, or biomaterial and conditioning, storage or delivery fluid, or biological extract and conditioning, storage or delivery fluid, or the conditioned biological extract alone. The biological assembly also comprises metabolites, growth factors, cells, exosomes, nucleic acids and other biological components produced during conditioning, if not specifically removed or exchanged.

### Conditioning Device

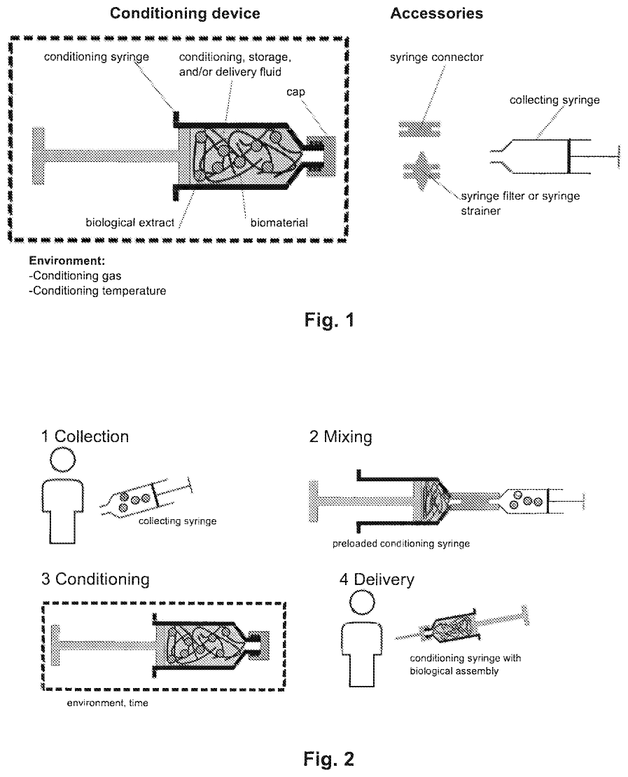

The conditioning device is represented in FIG. 1 (left side) and its accessories at the right side of FIG. 1. The conditioning device comprises a conditioning container. The condition container can be loaded with a biological extract, and contains or can be loaded with a biomaterial. When ready for conditioning the condition container comprises a biological extract, a biomaterial (e.g. hydrogel), a conditioning fluid (and possibly also a storage and/or delivery fluid.

Accessories to be used with the condition device can further comprise a collecting syringe or container for collecting a biological extract, a syringe or container connector for connecting the collecting syringe or container to the conditioning container, a syringe or container filter or syringe or container strainer, and static mixing components,

### Conditioning Container

The conditioning container enables conditioning of the biological assembly during a defined incubation time, and defined environment. It also has provisions for addition of biological extract, for instance by connection to a collection syringe or container, and provisions for delivery of the bioimplant obtained by conditioning biomaterial, biological extract and possibly conditioning fluid. In some embodiments, the conditioning device can also be the tissue collection device. The conditioning container is to be used sterile, disposable or re-usable. In case of re-usable accessories, they must be sterilizable.

### Conditioning Syringe or Container

The conditioning syringe or container is a preferred embodiment of the conditioning container. It enables conditioning of the biological assembly during a defined incubation time, and defined environment. It also permits injection of the biological assembly after conditioning, for instance by connection to an injection cannula. In some embodiments, the conditioning syringe or container can also be the collecting syringe or container. The conditioning syringe or container is to be used sterile, disposable or re-usable. In case of re-usable accessories, they must be sterilizable.

### Storage Fluid

A storage fluid is a medium of composition suitable with short, middle or long-term storage of the biological assembly.

### Conditioning Fluid

The conditioning fluid is a fluid chosen to permit desired conditioning of the biological extract. It is biocompatible at least under in-vitro conditions, possibly also under in-vivo conditions, and permits to achieve the targeted conditioning together with the environment.

### Conditioning

Conditioning is exposure to particular conditions, coming from the environment in which the conditioning syringe or container is placed and/or from the conditioning fluid to which the biological assembly is exposed during a certain time. The conditioning environment can use a particular gas composition, particular temperature, humidity conditions that will be permeating through the barrel wall of the conditioning syringe or container. Conditioning can consist of several steps with different gases, conditioning fluids and also involve mixing of differently conditioned fractions of identical or different source.

### Delivery Fluid

The delivery is fluid delivered within the biological assembly by injection or implantation. The delivery fluid is a biocompatible fluid, and can be identical to conditioning or storage fluid, or different.

### Biological Extract

The biological extract is conditioned through the use of the devices and methods of this invention. The biological extract can be from various sources such as the patient undergoing treatment, heterologous donors, of animal origin, or from cell-banked material, and can involve cells, but also extracellular matrix or cell fragments and vesicles.

### Biomaterial

The biomaterial is biocompatible in-vitro and optionally in-vivo, for example for in-vivo delivery. The biomaterial prevents sedimentation of the biological extract. For this, it can comprise a viscosing agent, a hydrogel, or particulate material, or combinations thereof. In embodiments where it is delivered in a patient, it is injectable.

### Growth Factor

A growth factor is a substance capable of stimulating cell proliferation, wound healing, regeneration and organ growth and function. Growth factors can also influence cell metabolism, gene expression, signalling, migration, differentiation and other cellular processes

### Accessory

Accessory are small utilities to perform accessory task such as to make connections between syringe or containers, cap syringe or containers, or filter syringe or container content. The accessories are sterile, and disposable or re-usable. In case of re-usable accessories, they must be sterilizable.

### Affinity

Affinity is the propensity of one substance to bind another substance. Affinity is typically measured by the dissociation constant K_{D}. In its simplest expression defined as the product of the free concentrations of the two substances of interest, divided by the concentration of the complex resulting from binding of the two substances, at thermodynamic equilibrium. In the case of a biomaterial interacting with a protein, the concentration of free protein is defined in the usual way, that is by the number of moles of protein molecules dissolved per volume of liquid. The concentration of biomaterial is taken as the concentration of repeat units per volume (if of unknown molecular weight, assumed to have a molecular weight of 200 g/mol), while the concentration of bound protein is the volumetric concentration of protein molecules tightly associated with biomaterial. In the case of a porous biomaterial, the concentration of bound protein can be approximated by its concentration in the wall material, while the free concentration is approximated by the protein concentration in the pore fluid.

### Pore Diameter

In a porous biomaterial, the pore diameter to wall thickness can be measured by confocal microscopy, using either autofluorescence of the wall material or specific staining (for example using 5 microgram/mL rhodamine 6G hydrochloride in deionized water for anionic polysaccharides). Algorithms for evaluation the pore size are well known. For example, after thresholding of digitized pictures representing confocal planes from confocal microscopy, the mean pore diameter can be evaluated by placement of maximal circles at every pixel in the pore space, followed by evaluation of their mean diameter with area weighting.

### Pore Diameter to Wall Thickness Ratio

In a porous biomaterial, the ratio of pore diameter to wall thickness can be measured by confocal microscopy, using either autofluorescence of the wall material or specific staining (for example using 5 microgram/ml rhodamine 6G hydrochloride in deionized water for anionic polysaccharides). Algorithms for evaluation of both pore size and wall thickness are well known. For example, after thresholding of digitized pictures representing confocal planes from confocal microscopy, both the mean pore diameter and the mean wall thickness can be evaluated by placement of maximal circles at every pixel in both the pore space and wall space, followed by evaluation of their mean diameter with area weighting. Once the mean pore diameter and the mean wall diameter evaluated, the calculation of their ratio is performed by division.

### Particle

A particle is a contiguous piece of biomaterial, typically in the size range of 10 nm up to many centimers. If the biomaterial is dispersed in an excess of solvent, individual particle will start to move as individual particles. The biomaterial may consist of one or a multitude of particles; in non-crosslinked biomaterials, no particles may be present, and molecules or molecular assemblies may be the largest contiguous unit.

### Swelling Capacity

The swelling capacity of a biomaterial is measured by dispersing a biomaterial in excess deionized water and then measuring the volume of the sediment. The swelling capacity can be quantitatively expressed as the sediment mass minus the original mass, divided by the original mass of biomaterial present. The swelling capacity is a dimension-less number, and is sometimes expressed in %. The notion of swelling capacity is directly applicable to soluble biomaterials-their swelling capacity is infinite as they will dilute to any available liquid volume.

### Young's Modulus

The Young's modulus is a well-known measurement of mechanical stiffness. It is measured by linearly compressing a sample of biomaterial, and is obtained as the slope of stress (force per area) vs. strain (relative compression compared to original height). For porous biomaterials, the Young's modulus is determined under drained conditions, i.e. provision is made to permit for timely escape of pore fluid. Drained conditions can be achieved by measuring the material in a submerged state, along with slow compression speeds (1 cm/min or less for biomaterial samples with heights on the order of a few mm).

### Viscosity

Viscosity can be measured with various types of commercially available viscometers and rheometers. The SI unit of dynamic viscosity is the newton-second per square meter (N·s/m²), also frequently expressed in the equivalent form of pascal-second (Pa·s). Some biomaterials have a yielding transition with liquefaction only above a certain shear stress. For such biomaterial, the viscosity is measured with shear stresses sufficient to induce yielding.

### Storage Modulus G′

The storage modulus G′, also known as the elastic modulus G′ is a well-known measurement of mechanical stiffness. It is measured in oscillatory shear, and corresponds to the in-phase component relating shear stress (force per area) to shear strain (relative shear strain). The shear modulus G′ of biomaterials is measured with low shear strains (0.1% to 1%), at sinusoidal oscillatory movement with a frequency of 0.2 Hz.

### Reversible Compressibility

Reversibly compressible means that the biomaterial can compressed at least by a specified factor factor (for example 2, 3 or 5), recovering at least 80%, preferentially 90% of the initial mechanical properties and namely the Young's modulus or elastic storage modulus G′ after release from the compression conditions.

### Partition Coefficient

The partition coefficient is the capacity of a biomaterial to bind a target factor. The partition coefficient for a given biomaterial and factor can be measured by confocal microscopy in the presence of fluorescently labelled factor as the ratio of fluorescence intensity between biomaterial and adjacent fluid, or in the case of a porous biomaterial, also pore fluid.

### Secretome

The secretome of a cell, collection of cells, or biological extract is defined as the entirety of secreted molecules. This includes proteins-growth factors, cytokines, extracellular matrix proteins, soluble receptors, hormones, paracrine factors, immune mediators such as interleukins, antibodies complement factors; hormone-binding proteins and other proteins. The secretome also includes partly proteic entities such as exosomes, lipoproteins, nucleoproteins, but also non-proteic components, such as neurotransmitters, paracrine factors such as prostaglandins, corticoids, steroids, membrane phospholipids and derivaties such as lysophosphatidic acid, eicosanoids, leukotrienes, lipoxins, platelet-activating factor, sphingosine 1-phosphate (S1P), and 2-arachidonoylglycerol and other lipid mediators, metabolites and signalling molecules such as ATP. ADP, adenosine and many others.

### DETAILED DESCRIPTION OF THE INVENTION

The basic workflow of the invention consists in assembling a bioimplant, conditioning it, and reimplanting it. The condition step is performed in a gas permeant conditioning container, in the presence of a biomaterial mixed with the biological extract of interest (FIG. 1).

The method of use according to the invention consist in four basic process steps, namely (i) the collection of the biological extract, (ii) the mixing of the biological extract with a biomaterial and a medium, (iii) the conditioning of the mixture obtained in step (ii), and (iv) the delivery of biological assembly obtained by the conditioning step (iii).

A prototypical method of use of the invention is outlined in FIG. 2.
1) The first step is the collection of the biological extract.
2) This is followed by mixing the biological extract with the biomaterial and conditioning media in the conditioning syringe or container
3) The third step is conditioning within the conditioning syringe or container, placed within the conditioning environment.
4) The biological assembly obtained in this manner is then delivered, for example by injection in a patient.

The elementary method of use is shown in FIG. 2 and is ideally, but not necessarily, performed intraoperatively in a closed system. Isolation typically involves surgical intervention and preparation of clinical samples such as lipoaspirate, blood plasma, biopsies and the like. These isolates are then mixed with a preloaded incubation syringe or container or other container according to the invention. Thorough mixing is typically obtained by joining the preloaded syringe or container and a second syringe or container containing the isolate via suitable plastic pieces (Luer-Luer connection, for example), and passing the mix several times through the connection. To facilitate mixing, static mixing structure can be present either in the container or the connection; such static mixing structures are known in the art (e.g. U.S. Pat. No. 3,286,992). After loading, the living isolate is conditioned by the combination of media elements already contained in the preloaded syringe or container, and the controlled gas atmosphere by means of diffusion, and temperature and time. Once the mixed material conditioned, it can be injected into patients, typically the same patient from which the isolate was obtained (autologous) or a compatible recipient (heterologous).

The biomaterial can be polymer-based. Polymer-based biomaterials are preferably chosen form the following groups of substances;
Hydrogels, in particular:
extracellular matrix-based hydrogel, including collagen, elastin, matrigel, laminin, fibrinogen, fibrin, fibronectin; hyaluronic acid-based hydrogel, glycosaminoglucane based hydrogels, including chitosan-based hydrogel; acrylates; cellulose and its derivatives including carboxymethylcellulose, hydroxyethylcellulose, hydroxypropylcellulose, methylcellulose; agarose; alginate; gelatin; heparin, heparosan, nucleic acids, DNA, RNA
other polymer-based biomaterials, in particular:
silk-based biomaterial; decellularized plant- or fungus-based biomaterial; synthetic polymer including polyurethanes, silicones, polyolefins, poly-vinyl-alcohol, polyamides, polyimines, polyethers, polyethyleneglycol and mixtures thereof.

In a preferred embodiment, the biomaterial is a crosslinked polymer-based biomaterial, obtained by covalent or non-covalent crosslinking or a combination thereof. Examples of crosslinkers are bi- or multifunctional amines for amine formation, bi- or multi-functional epoxides, epihalohydrins, bi- or multifunctional halides via Williamson ether synthesis, thiol-based crosslinking via disulfide bond formation or thiol-ene reaction, genipin, click chemistry (Diels-Alder, reverse electron-demand Diels-Alder, azide-yne reactions), oxidative crosslinking of catecholamine groups, Schiff-base crosslinking, formaldehyde or bi- or multifunctional aldehyde crosslinking, reductive amination, urethane formation from hydroxyl and isocyanate groups and other known means. In another embodiment, the covalent crosslinking is achieved through the crosslinking of native groups of the backbone of the polymer.

In a preferred-embodiment, the biomaterial is porous. It may consist of a single contiguous piece of biomaterial or of a suspension of porous particles. Porous biomaterials can be produced by various means in the art, for example using porogen leaching, gas foaming, cryopolymerization, lyophilization, emulsion polymerization, or thermally induced phase separation. The pore fraction is advantageously in the range of 30% to 99%, more preferentially 50% to 95%; the pores are preferentially interconnected, with an interconnected pore space advantageously representing more than 50%, more preferentially more than 70% of the total pore space. Pore fraction and interconnectivity can be measured through the wicking test (Beduer et al., Adv Healthcare Materials 10.1002/adhm.201400250).

In a preferred embodiment, the particles have protrusions at their surface or within the particle itself. In a preferred embodiment, the protrusions size represents 10% and 500% of the particle size, preferably between 20% and 100%.

In a special embodiment the syringe or container may also comprise ceramics, preferably hydroxyapatite-based biomaterials and/or calcium phosphate.

The biomaterial can also be based on extracellular matrix. The extracellular matrix can be obtained by decellularization of various organs and tissue extracts, for example skin, lipoaspirate, excised adipose tissue, muscle tissue, muscle tissue, heart tissue, meat, artificial meat, nerves, vascular tissue and others. Purified extracellular matrix components such as collagen, elastin, laminin, fibrin or fibronectin can also be chosen.

The biomaterial can also be patient-derived. Patient-derived materials may for instance be chosen from the group of lipoaspirate, bone marrow extract, bone fragments, blood, plasma, platelet-enriched plasma, fibrin and coagulated blood.

Mixtures of polymer-based materials, extracellular matrix material and patient-derived materials can also be used.

In a special embodiment the overall oxygen permeability of the container is at least 1 mL/Pa/m²/day and preferably 10 ml/Pa/m²/day.

In a further embodiment the viscosity of the biomaterial is above 0.02 Poise, preferentially, 0.1 Poise and preferably above 1 Poise.

The container, preferentially a syringe or container, may comprise an amount of biomaterial corresponding to 0.01%-99.00% of the container volume, so that the container is not entirely filled.

The amount of biomaterial comprised in the container corresponds purposefully to 0.1%-80.0% and preferably to 1%-50% of the container volume:

According to a special embodiment the biomaterial-preferably a hydrogel—is porous and reversibly compressible by at least a factor of 2, more preferentially 3 or more. Reversible compressibility is achieved through sufficiently high pore diameter-to-wall thickness ratios. Advantageously, the pore diameter to wall diameter is above 1, more preferentially above 2, even more preferentially above 3 and most preferentially above 5. The pore diameter itself is preferentially in the range 20 micrometers to 5 mm, more preferentially above 40 micrometers to 2 mm, and most preferentially in the range of 50 micrometers to 1 mm. The pore diameter is important both for retention of the biological extract in a homogeneous state in the conditioning container, and subsequent in-vivo reaction. Pore diameters above 20 micrometers, more preferentially 40 micrometers, and most preferentially above 50 micrometers are more favorable to vascularization, colonization and regeneration.

Preferentially, the porous biomaterial is contained in the syringe or container in a state of partial hydration, or dry, and is capable of absorbing substantial amounts of supplementary liquid, Preferentially, the biomaterial is capable of absorbing at least 10% of its own volume, more preferentially 20% of its own volume, even more preferentially 50% of its own volume, and most preferentially 100% of its volume or more of cell extract. The capacity of a biomaterial to absorb liquid by swelling can be quantified by its swelling capacity, which should be at least 10%, preferentially 20%, even more preferentially 50% and most preferentially 100% or more. This enables the biomaterial to swell sufficiently to absorb the entire amount of biological extract by swelling, thus forming a homogeneous semi-solid or gel-like suspension, keeping the components of the biological extract in place during the conditioning period. This is further aided by the preferential porous nature of the biomaterial, permitting to draw the components of the biological extract into the pore space during expansion of the biomaterial to occupy the additional volume added by the addition of the extract.

According to a further embodiment the conditioning container may further comprises an amount of conditioning fluid. In a preferred embodiment, the condition fluid comprises 0.1 mM to 3 mM of glucose or of 2-deoxy-D-glucose. This induces pro-angiogenic molecules, to induce blood vessels once implanted. For this type of application, the conditioning amounts to starvation and hypoxia.

In some embodiments, the conditioning fluid may contain biologically active concentrations of one or several of the following substances: insulin and insulin analogs, insulin-like growth factor, analogs and activators and analogs of AMPc such as 3-Isobutyl-1-methylxanthine IBMX, short chain fatty acids such sodium butyrate or propionate or other ketone bodies, fatty acids such as oleic acid, glucocorticoids such as dexamethasone, thiazolidinediones such as rosiglitazone or pioglitazone, sulfonylureas such as glimepiride, beta-blockers, anti-seizure drugs as pregabaline. This special embodiment applies molecules inducing adipogenic differentiation, in an environment providing excess of nutrients. While it is difficult to apply both simultaneously (“they are at odds”), what can actually be done is to separate the surgical material into two fractions, apply the two conditioning methods separately, and remix the two fractions before applications in order to obtain vascularization from the stressed part, and then adipose differentiation from the primed stem cells.

The conditioning fluid may have a glucose content from 4 mM to 50 mM and preferably from 6 mM to 20 mM.

The conditioning fluid may contain an iodine-based disinfect, preferably a povidone-iodine, with an effective iodine concentration from 0.1 mg/mL to 11 mg/ml, more preferentially from 0.3 mg/ml to 3 mg/mL.

The conditioning fluid may further comprise one or more of the following substances: dimethyl sulfoxide, glycerol, mannitol, trehalose, serum (autologous, heterologous, or xenologous), blood plasma or polyvinyl alcohol, in concentrations appropriate for cryoprotection.

The volume of conditioning fluid plus hydrogel purposefully is between 0.01% and 99% of the syringe or container volume, preferentially between 0.1% and 80%, more preferentially between 1% and 50%.

The conditioning fluid may contain effective amounts of cryoprotectants such as dimethylsulfoxide, glycerol, mannitol, trehaolose or other small molecules suitable for cryopreservation, and/or cryoprotectants with macromolecular constituents such as serum (autologous, heterologous, or xenologous), blood plasma, or polyvinyl alcohol.

The volume of the syringe or container may be comprised between 0.1 ml and 500 mL, preferentially 0.5 mL-100 mL, more preferentially 1 mL-30 mL]

The Invention is also directed to an assembly comprising the conditioning container, preferentially in the form of a syringe or container according to the invention and a closable container designed to lodge the syringe or container therein. In a special embodiment of the assembly (I) the syringe or container is housed in the interior of the container; and (ii) the container comprises a controlled gas composition with an amount of oxygen.

The gas composition of the container purposefully comprises between 0.01% and 10% of oxygen.

In a special embodiment the gas composition of the container comprises between 0.1% and 5%, and preferably between 0.5% and 2% of oxygen

The gas composition may further comprises between 2 and 15%, preferably between 5% to 10% of carbon dioxide.

The invention is also directed to a method for obtaining a conditioned biological assembly, consisting of:
mixing a polymer-based biomaterial and a conditioning fluid contained in a conditioning syringe or container with a biological extract; and
conditioning of the biological assembly thus obtained in a conditioning environment for an incubation time from about 5 minutes to 12 hours, for obtaining a biological assembly for minimally invasive delivery.

The conditioning fluid medium and the biomaterial are compatible with the survival and conditioning of living elements of the biological extract, for an incubation time from about 5 minutes to 12 hours, more preferentially from 15 minutes to 5 hours, and most preferentially from 30 minutes to 3 hours.

In a special embodiment of the method according to the invention the incubation time is from 15 minutes to 5 hours, and preferably from 30 minutes to 3 hours.

In a further embodiment the method further comprises the steps of:
(I) transferring the syringe or container with the biological assembly into a closable container comprising a gas composition with a concentration of 0.01-10% of oxygen; and
(ii) incubating the syringe or container for 5 minutes to 12 hours at a defined temperature from 34° C. to 40° C. while the oxygen of the container continuously permeates through the gas permanent barrel wall of the syringe or container.

The biological assembly obtained can be used either in vitro or in vivo.

An important role of the biomaterial is to maintain and reinforce the secretome accumulated during conditioning. For this, the partition coefficient of target growth factors between biomaterial bound fraction and free fraction is of prime importance. Target growth factors of interest include for example factors chosen within the group of VEGF-A, IGF-1, FGF-1, FGF-2, PDGF, HGF, KGF, SDF1, TGFbeta, EGF, insulin and adiponectin, Other target factors can be NGF, BDNF, GDNF, VEGF-C, TGF-alpha, TNF-alpha, BMP-2, BMP-4. Further factors of interest can be interleukins, chemokines, interferons, antibodies, exosomes, complement factors and namely C1r, C1s and C1q. Target factors can also hormones or paracrine substances such as prostaglandins PGE2 or PGI2, The partition coefficients for factors of interest advantageously lies in the range of 1 to 1000, more preferentially to 2 to 500 and most preferentially 5 and 250 when comparing the concentrations in the wall fraction of porous biomaterials as compared to the pore fluid of said porous biomaterials.

The targeted biomaterial's partition coefficient for a specific targeted factor can be achieved by functionalizing the biomaterial with specific binding molecules, Such binding molecules can be antibodies, fragments of antibodies, minibodies, peptides, receptors, fragments of receptors self-folding complementary RNA or DNA molecules, substrates, or any other chemical entity with affinity for the factor of interest.

Functionalization can be achieved by means known in the art, for example through amide or ester bond formation from activated esters or through the action of carbodiimide, Michael addition of thiols, oxidative coupling of thiols via disulfide bridge formation, hydrazone formation, imine formation, click chemistry (for example azide-aryne, thiol-ene, Diels Alder, Staudinger ligation, inverse electron-demand Diels-Alder) and other conjugation techniques known in the art.

The targeted biomaterial's partition coefficient for a specific targeted factor can be also be achieved by adjusting the composition of the biomaterial.

After implantation in a patient, the stored targeted factors will be released. At the very least, this concerns the factors physically held in the pore fluid and/or biomaterial. This permits to efficiently transfer accumulated secretome. The partition coefficient of the biomaterial for the accumulated secretome will dictate the release behavior.

The conditioning fluid may be exchanged, either between incubation periods, or after incubation and prior to delivery. Affinity-based retention of growth factors or other mediators is advantageous in this case to avoid dilution and washout of target growth factors of interest.

The biological assembly may be frozen for storage and thawed before minimally invasive delivery of the biological assembly to a patient.

The conditioning fluid may be exchanged by reversible compression of a porous biomaterial, preferably a hydrogel within the biological assembly.

According to a further embodiment the conditioning fluid contained in the pores of the reversibly compressible biomaterial within the biological assembly is at least partly removed by compression of the biomaterial by means of an appropriate plunger movement, and where the removed liquid is replenished with a new one by actuation of the plunger in the opposite direction.

The conditioning syringe or container may be closed with a cap that has a mesh with openings smaller than the particle size of the biomaterial, and then slowly actuating the piston of the syringe or container to remove pore fluid from the biomaterial until most of pore fluid is removed, before aspirating new, preferably different fluid, by inverse motion of the piston of the syringe or container.

In a further embodiment the method a further process step of decellularization of the biological assembly is performed after conditioning by exposure to a basic solution containing sodium or potassium hydroxide, or sodium or potassium phosphate, or other buffer, at a pH of at least 12, for at least 10 minutes, and where the decellularization solution is brought into contact with the biological assembly by the process step of fluid exchange.

The decellularization may be achieved through the use of anionic, cationic or neutral detergents or combinations thereof at an overall concentration of at least 0.1% for at least 10 minutes.

In a further embodiment a further process step after conditioning may be performed which consists of sterilization of the conditioning syringe or container and biological assembly contained therein by exposure for at least 10 minutes to at least 110° C. under pressure and steam atmosphere.

In another embodiment of the method a further process step after conditioning is performed and consists of sterilization of the conditioning syringe or container and biological assembly contained therein by exposure to a gamma irradiation to a minimum of 25 kGy.

In still a further embodiment of the method a further process step after conditioning is performed and consists of sterilization of the conditioning syringe or container and biological assembly contained therein by exposure to a gamma irradiation at least to a level capable of achieving a sterility assurance level of 10-6 as extrapolated from exponential decay of life bacteria under lesser doses and the count of live bacteria at the onset of sterilization.

In a special embodiment of the method according to the invention fractions of the biological extract are placed in different syringe or containers and conditioned differently, before being recombined.

The Invention is directed also to a biological assembly comprising the following components:
a biological extract modified by exposure to controlled hypoxia and containing at least 1 ng/ml of vascular endothelial growth factor A (VEGFA); and
a hydrogel suitable for minimally invasive delivery

The biological assembly may be contained in a device compatible with injection or minimally invasive delivery, obtained by one of the methods according to the invention. The biological assembly can be applied to various treatments/applications, e.g.
for constructing, reconstructing or enhance adipose tissue;
for improving skin quality after radio- or chemotherapy;
for enhancing the outcome of dermal filler injection or in conjunction with bioprostheses, in particular with silicone implants; or
for enhancing wound healing, or vascularization, in particular in arterial insufficiencies of the lower members or in diabetic patients.

It is easily understood that there are many variations and adaptions of this basic method, and important aspects of the methods of use are better understood through the description of the following three use-cases.

### Example No. 1: Intraoperative Pre-Conditioning of Surgical Isolates

In this use case, illustrated in FIG. 2, a surgical isolate is collected by established means with a collecting syringe or container (step 1 Collection). For example, lipoaspirate can be obtained by aspiration with a liposuction canula, optionally supported by shear movement, ultrasound, liquid injection or pharmacological means such as treatment with adrenaline. The lipoaspirate thus obtained can be used as is or processed further by mincing, centrifugation, separation or other means known in the art, and stored in a collecting syringe or container.

The lipoaspirate is the biological extract to be treated. Once ready for processing with the present invention, the collection syringe or container and a preloaded conditioning syringe or container are connected for mixing, for instance using a luer-luer connection (step 2 Mixing shown in FIG. 2). The conditioning syringe or container is pre-loaded with a concentrate of biomaterial and conditioning medium, the biomaterial having the capacity to absorb the liquid volume of the lipoaspirate. This can for example be achieved by a non-porous biomaterial consisting of particles capable of absorbing part of the additional by swelling, or by a porous, elastic biomaterial capable of elastically swelling while taking up the additional volume. Alternatively, a phase-transition material can be used, for example methylcellulose or PNIPAAm-based materials that are viscous liquids while at lower temperature but gel will later gel once the conditioning is started with elevation to higher temperature, or also agarose that transitions from a viscous, liquid state to a gel upon cooling to conditioning temperature. In any case, homogenization is achieved either by swelling of the preloaded biomaterial in contact with the additional material alone, or by mechanical mixing, or both. Other methods such as aspiration or loading of syringe or containers from a larger, stirred mix are possible as well, and also distribution from a given collection syringe or container to multiple conditioning syringe or containers, or inversely, combination of the content of multiple collection syringe or containers into a given collection syringe or container. It is also possible to load a conditioning syringe or container with several biological extracts, simultaneously or sequentially, for instance with lipoaspirate and separately enriched stromal vascular fraction, or lipoaspirate and autologous or heterologous platelet-rich plasma, or lipoaspirate and mobilized hematopoietic stem cells, or lipoaspirate and autologous or heterologous serum. Off-the shelve components such as cultured mesenchymal stem cells or cold-stored, thawed samples can be used as well, in combination with other biological extracts or alone.

Once a homogeneous biological assembly is obtained in the conditioning syringe or container, the conditioning syringe or container is placed in the environment of a container (at a controlled conditioning temperature and containing a conditioning gas) to carry out the conditioning step (as shown in step 3 Conditioning of FIG. 2). In intrasurgical use for lipoaspirate processing, the environment can be an incubator with controlled oxygen level, humidity, and possibly carbon dioxide levels, at a temperature typically near body temperature. To enhance the vascularization potential, incubation is performed in conditions of lowered oxygen partial pressure, or in conditions of metabolic starvation, or both. The oxygen tension is controlled through diffusion through a gas permeant syringe or container material. Typical choices of syringe or container material are thin membranes of silicones, silicone copolymers, fluorated polymers, or porous hydrophobic membranes. Gas permeable syringe or container constructions are to be described further on. To enhance vascularization, low oxygen conditions (typically between 0.01% and 5%, more preferentially between 0.1% and 1% of oxygen in the gas phase at normal atmospheric pressure) are known to induce pro-angiogenic signals in many cell types, with enhanced expression of growth factors such as VEGF-A, Low nutrient conditions (for example, 0 to 4 mM of glucose, more preferentially 0.1 mM to 3 mM and even more preferentially 0.5 mM to 2 mM), lack of non-essential amino-acids, addition of glucose antimetabolites such as 2-deoxy-D-glucose and others are also able to promote expression of pro-angiogenic factors and certain immunomodulatory molecules.

Moderate hypoxia and low glucose levels can be combined. To enhance adipogenic differentiation, adipogenio differentiation factors can be added-insulin and insulin analogs, insuline-like growth factor, analogs and activators of AMPc such as 3-Isobutyl-1-methylxanthine IBMX, short chain fatty acids such sodium butyrate or propionate or other ketone bodies, glucocorticoids such as dexamethasone, thiazolidinediones such as rosiglitazone or pioglitazone, sulfonylureas such as glimepiride, beta-blockers, anti-seizure drugs as pregabaline and others being examples. The glucose content can also be increased to supraphysiological levels, for example from 5 mM to 50 mM, preferentially from 6 mM to 20 mM. Combinations of mediators and conditions are possible. Conditioning takes place for a defined period of time, from about 5 minutes to 12 h, more preferentially from 15 minutes to 5 h, and most preferentially from 30 minutes to 3 h.

After conditioning, in the simplest case, the conditioning syringe or container is used for minimally invasive delivery of the conditioned biological assembly contained therein (step 4 Delivery shown in FIG. 2). For this step, techniques known in the art, such as hypodermic needles or delivery cannulae screwed or clipped to the syringe or container tip can be used. While in some embodiments, the dose of active agents added to the conditioning fluid is unproblematic and can be injected along with the conditioned biological assembly. In other embodiments, the delivery medium is distinct from the conditioning fluid and medium exchange is necessary. This is discussed in detail in other use cases. The conditioned biological assembly can be implanted minimally invasively for various purposes, for instance to construct, reconstruct, or enhance adipose tissue, to improve the quality of the skin after radio- or chemotherapy, to enhance the outcome of dermal filler injection or in conjunction with bioprostheses such as silicone implants, to enhance wound healing, to enhance vascularization, particularly in arterial insufficiencies of the lower member or in diabetic patients or both, for re-injection to enhance the effect of prior dermal filler or other regenerative material injection and other applications.

### Example No. 2: Combined Collection and Conditioning Syringe or Container

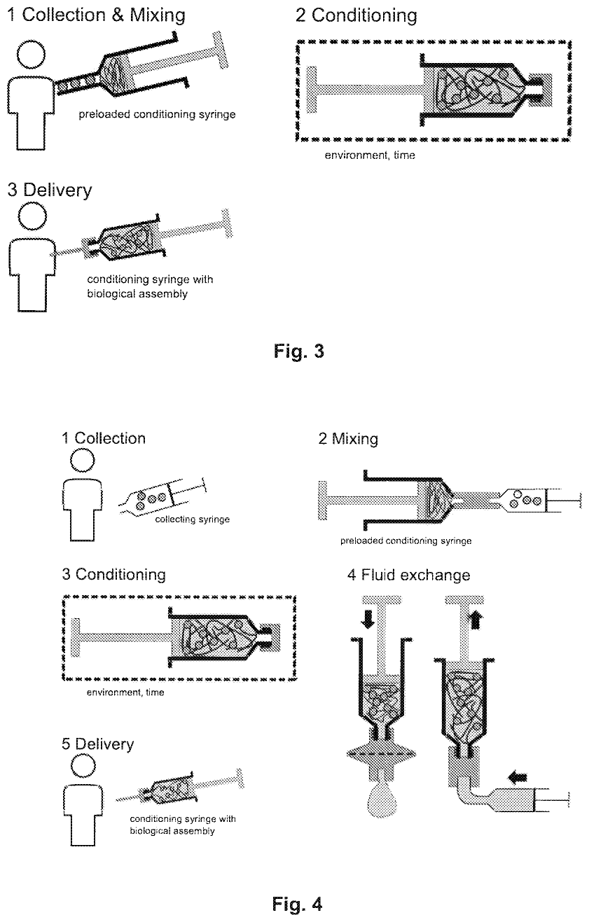

Use case no. 2, as illustrated in FIG. 3, distinguishes itself from use case no. 1 in that the conditioning syringe or container also serves as a collection syringe or container. The mixing of the biomaterial, conditioning fluid and biological extract happens directly when collecting the biological extract in the conditioning syringe or container (step 1 Collection & Mixing shown in FIG. 3). In this case, the use of a reversibly compressible biomaterial, as taught for example in WO2017029633A1, is particularly useful, as the swelling of the biomaterial will draw the additional liquid volume acquired with the biological extract into the biomaterial. Mechanical mixing is alternatively possible, although this generally requires the use of an additional syringe or container to pass the biological assembly between the two syringe or containers, or the introduction of a stir mechanism into the syringe or container. Alternatively, particulates of phase change materials can also be used, for example methylcellulose gel in a preheated syringe or container where for dissolution, the conditioning syringe or container is transiently placed in a lower temperature environment, for example at 4° C. or 20-25° C. In this case, mixing is achieved by rapid indraw of biological extract without dissolution of the particulate, permitting homogenization. Upon transiting to the lower temperature, the particulate dissolves and creates a viscous liquid that further stiffens upon return to body temperature during conditioning. A combination of reversibly compressible particles and temperature-induced phase change is also advantageous. In this case, the reversibly compressible particles are filled with a cold-solidifying gel in a compressed state, and the mass mechanically fragmented to achieve an overall flowable nature. This can for example be achieved by immersing porous elastic particles as taught by WO2017029633A1 in a compressed state in a small amount of hot, liquid gelatin solution (10%), followed by solidification at lower temperature (4° C.). Upon fragmentation of the mass at low temperature, a collection of distinct particles is obtained. These particles can be mixed with biological extract, including at room temperature, but when heated to 37° C., the gelatin becomes liquid and permits expansion of the compressed particles while drawing in the biological extract, thus generating a homogeneous mixture stabilized by the expanded state of the reversibly compressible particles, with the gelatin diluted in the larger liquid volume. A similar approach can be used with substances such as methylcellulose or PNIPAm (poly N-isopropylacrylamide, optionally as co-polymer with acrylic acid or acrylamide to adjust melting temperature), but in this case the compressed state is fixed at higher temperature with particle expansion freed upon lowering the temperature.

Aside from the combination of steps 1 and steps 2 of use case no. 1 (collection, mixing) into a single combined step, the remainder of the use case no. 2 (step 2 Conditioning and step 3 Delivery) can be contemplated from use case no. 1.

### Example No. 3: Fluid Replacement

Use case no. 3, illustrated in FIG. 4, adds a step of fluid exchange (step 4 Fluid exchange). Fluid exchange is necessary in several use case. For instance, the conditioning media may contain substances that are necessary to achieve the desired metabolic or differentiation conditioning during the conditioning step, but present too high levels of toxicity or other undesirable effects for implantation. Fluid exchange is achieved by squeezing out fluid from the biological assembly while maintaining the relevant proportions of the biological assemble in place. A syringe or container filter (pore size in the range of 50 nm to 500 micrometers) or syringe or container strainers (larger mesh pore sizes, up to 1 mm) can be used for this purpose. For fluid exchange, not all biomaterials are equally suitable. Reversibly compressible, porous biomaterials are particularly suitable, such as the ones taught in WO2017029633A1. By actuating the plunger, fluid can be extruded through the syringe or container filter, while maintaining the material in place. If fluid extrusion is carried sufficiently slowly (1 min to 2 h, preferentially 5 minute to 1 h, most preferentially 10 minutes to 30 minutes), cells will remain in place on the biomaterial, including in the absence of adhesion motives, due to spatial entrapment. Rapid fluid extrusion on the other hand can be leveraged to achieve cell removal, if so desired. New fluid can be added for instance by connecting a syringe or container containing the new fluid through a syringe or container-syringe or container connector, or tubing, or both, as shown in FIG. 4. Alternatively, new fluid can be drawn Into the syringe or container from a reservoir, added via a syringe or container pump, injected via a cannula through the opening of the syringe or container or by any means known in the art.

Fluid exchange can be used to add delivery fluid in exchange of conditioning fluid as described. Other purposes can be pursued and are various. Decellularization conditioning fluids can be used for retrieval of matrix components on the biomaterial in the absence of cellular components. Many decellularization compositions are known in the art, they typically include surfactants, but may also include strong base, DNases, other enzymes, complexing agents such as EDTA and other substances. Storage fluids can also be introduced by fluid exchange, for instance for cryoprotection. Fixing conditioning fluids, for example based on ethanol, formaldehyde, glutaraldehyde or other fixing agents well known in the art can be used to fix the biological assembly and also inactivate pathogens or bacteria. Fixing and decellularization solutions are toxic and thus need to be replaced by delivery medium, and so do certain cryoprotectants, for which fluid exchange can again be used.

### Example No. 4: Revascularization of Diabetic Ulcer

1. Extraction of Tissues from the Patient:

Liposuction to aspirate 100 ml of adipose tissues and centrifugation to collect stromal vascular fraction.

### 2. Creation of Biological Implant:

Inject of 3 mL of the SVF into a bag recipient containing through the injection port:
10 ml of a biomaterial made of crosslinked cellulose scaffold partially hydrated (60% initial compression, buffered at pH. 5), 80% porosity, of which 75% interconnected
7 ml of cell culture medium including 30 mg/g of fibrinogen.

Close the port of the bag.

### 3. Conditioning of Biological Implant:

Place the closed recipient bag in an incubator with 5% CO₂, 5% Oz and 37° C. during 12 hours.

### 4. Delivery of the Conditioned Biological Implant:

Add thrombine solution, to a final concentration of 0.5 U/mL, and CaCl₂ to final concentration of 1.5 mM, and mix by gently squeezing the bag during 2 minutes.

Connect the cutaneous applicator to the recipient bag and apply the biological implant on the surface of the ulcer.

In a last step, the ulcer is covered using standard of care practices.

### Example No. 5

A solution consisting of 25 g deionized water, 4.5 g acrylamide, 0.25 g sodium acrylate and 0.15 g bisacrylamide is prepared. To this solution, 25 microliters of 10% tetramethylethylenediamine and 25 microliters of 10% ammonium persulfate solution are added. Before polymerization occurs, 100 g sodium chloride powder is added and the slurry resulting slurry is let to polymerize overnight. The resulting polymer is ground to about 1 mm particles during dissolution. Thanks to presence of acrylate groups, the resulting porous particle slurry has displays a partition coefficient of about 100 for FGF-2 at pH 7.4 in phosphate buffered as measured by confocal microscopy with rhodamine-isothiocyanate-labelled FGF-2. Upon regular replenishment of buffer, release of 50% of the bound FGF-2 occurs within 1 week.

### Example No. 6

Heparin sodium salt, 1 mg/ml, is covalently linked to porous adipic acid crosslinked alginate (3%, synthesis according to 10.1002/adhm.201400250) by using 1-Ethyl-3-(3-dimethylaminopropyl) carbodiimide hydrochloride, 10 mg/mL as an activator for ester bond formation. As assessed by confocal microscopy with rhodamine-isothiocyanate labelled VEGF-A, the resulting complex binds VEGF-A with a partition coefficient of about 1000. At 37° C. and a pH of 7.4, 50% of the VEGF-A is released within 1 month upon regular replenishment of the buffer solution. Aminofluorescein labelled heparin soldium salt used as a tracer is found to be released with similar kinetics. The hydrolytic lability of the attachment of the affinity-conferring agent is judged to be controlling the release kinetics in this case.

### Detailed Description of Single Elements of the Invention

### a) Regarding the Environment

An important aspect of the conditioning environment is gas phase composition. The conditioning gas may for instance contain defined concentrations of carbon dioxide CO₂, oxygen O₂, inert gases such as nitrogen Na or noble gases, nitrogen oxides NO, medical gases such as N₂O, and various mixes thereof. Humidity can also be controlled via the partial pressure of water vapor. In one embodiment, the conditioning gas is air with ambient composition. In another embodiment, the conditioning gas contains between 0.01% and 10% of oxygen, more preferentially between 0.1% and 5%, and most preferentially between 0.5% and 2%. In one embodiment, the conditioning gas contains between 2% and 15% of carbon dioxide.

The environment typically also involves a controlled temperature, for instance around-80° C. for transport, −196° C. for storage, −20° C. to −10° C. for freezing, 4° C. or room temperature (20-25° C.) for short-term storage. Most typically, incubation will take place around body temperature, i.e. at 37° C. (33-40° C., more preferentially 35-38° C.). In some embodiments, the environment is provided by a portable, sterilizable, closed incubator set to regulate the environment at the desired gas composition, humidity and temperature parameters.

In another embodiment, the environment provides mechanical conditions, such as mechanical agitation, stirring, centrifugation, rotation, microgravity.

In another embodiment, the environment provides UV irradiation, gamma irradiation in doses ranging from 1 Gy to 250 Gy for some indications such as cellular inactivation, or from 1 kGy to 100 kGy for other indications such as sterilization.

### b) Regarding the Biological Assembly

A biological assembly is produced through the use of the teachings of this invention, and is destined to be injected or implanted into a patient. The biological assembly typically consists of the biomaterial, the biological extract, and conditioning, storage or delivery fluid, but it may also consist of parts of the contents of the conditioning syringe or container. This can be biomaterial having undergone contact with the biological extract alone, or biomaterial and conditioning, storage or delivery fluid, or biological extract and conditioning, storage or delivery fluid, or the conditioned biological extract alone. The biological assembly also comprises metabolites, growth factors, cells, exosomes, nucleic acids and other biological components produced during conditioning, if not specifically removed or exchanged.

### c) Regarding the Conditioning Syringe or Container

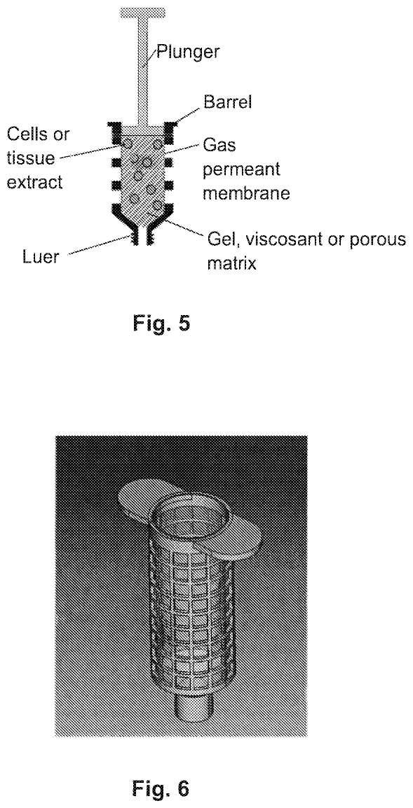

The gas-permeant conditioning syringe or container, as schematically shown in FIG. 5, comprises a barrel, plunger and luer. The wall of the barrel has special construction comprising two layers of different materials, one of which being a gas permeant membrane.

The barrel is filled cells or tissue extract and a gel (hydrogel), viscosant or porous matrix.

The gas-permeant conditioning syringe or container permits conditioning of the biological assembly during a defined incubation time, preferentially from 5 minutes to a month, more preferentially from 10 minutes to a day, and most preferentially from 15 minutes to 5 hours. In a preferred embodiment, the conditioning syringe or container is gas permeant, having an oxygen permeability of at least 0.01 mL/Pa/m²/day, and more preferentially 0.1 mL/Pa/m²/day, even more preferentially 1 mL/Pa/m²/day and most preferentially 10 mL/Pa/m²/day. Preferentially, it is also permeant to carbon dioxide. Despite being gas permeant, the conditioning syringe or container is typically chosen to be impermeant to living microorganisms and optionally viruses and thus maintains a sterile environment in its interior.

In the 3D view (FIG. 6) of the gas-permeant syringe or container, the cells, gel and plunger have been removed for better visibility. The barrel, constructed with openings, backs a thin, gas-permeant membrane or lining, made from silicones, track-etched hydrophobic membranes such polytetrafluoroethylene, or perfluorated polymers. The volume of the conditioning syringe or container is typically comprised between 0.1 mL and 500 mL, preferentially 0.5 mL-100 mL, more preferentially 1 mL-30 mL.

### d) Regarding the Storage Fluid

Storage fluids are can be formulated to enable low-temperature storage (−80° C., or liquid nitrogen temperatures) and for this aim may contain cryoprotectants such as dimethylsulfoxide, glycerol, mannitol, trehaolose or other small molecules suitable for cryopreservation, but also protectants with macromolecular constituents such as serum (autologous, heterologous, or xenologous), blood plasma, polyvinylalcohol and others. The storage fluid can also be a gas, such as ambient air or nitrogen or another inert gas, when the biological assembly is lyophilized for long-term storage.

### e) Regarding the Conditioning Fluid

The conditioning fluid is a fluid chosen to permit desired conditioning of the biological extract. The storage fluid can be a cell culture medium, differentiating medium, dedifferentiating medium, transdifferentiating medium, enriched medium, conditioned medium, platelet enriched plasma. It can also contain, or be enriched in disinfectants, particularly iodine-based disinfectants, preferentially at a concentration and duration combining antimicrobial activity and biological extract survival, contrast agents, antibiotics, pharmacologically active agents, viscosing agents and particularly methylcellulose, carboxymethylcellulose or hyaluronic acid, liposomes, free fatty acids, aminoacids, exosomes, growth factors, hormones, nucleic acids, peptides and more. The conditioning fluid may also be formulated to impose particularly low concentrations of certain metabolites, particular glucose and amino-acids, or contain particularly high concentrations of waste products such as lactate or ammonium. Regarding conditions suitable for conditioning cells to implantation, metabolic, immunological, and functional aspects can be tailored.

In one embodiment, the conditioning medium contains glucose, amino-acids, vitamins (B group, optionally vitamin C, optionally vitamin A or other retinoids, optionally vitamin E or anti-oxydants in the form of reducing agents such as N-acetylcystein, beta-mercaptanol, dithiothreitol, reduced glutathione), mineral salts providing for buffering capacity and osmolarity, and a variety of salt of oligoelements (iron, selenium, molybdenum, vanadium, zinc, copper, and others, optionally an organic source of phosphate such as beta-glycerophosphate, optionally also specific lipids such as oleic acid. An example of conditioning medium aiming at using partial starvation to induce secretion of pro-regenerative and pro-angiogenic factors would be Dulbecco's Modified Eagle's Medium DMEM with 10% autologous serum but without glutamine. Agonists of beta adrenergic receptors can be added here, particularly beta-3 activators such as Disodium-5-[(2R)-2-[(2R)-2-(3-chlorphenyl)-2-hydroxyethyl]amino}propyl]-1,3-benzodioxol-2,2-dicarboxylate Hydrate (CL316243, 1-50 nM). The conditioning medium can have a glucose concentration adjusted such that after mixing with the biological extract, the final glucose concentration is 2.5 mM (0.1 mM to 3 mM). In one embodiment, this conditioning medium is used for incubation in moderate hypoxia, i.e. for incubation of the biological assembly containing a porous, reversibly compressible hydrogel based on crosslinked carbohydrates such as hyaluronic acid, heparosan, chitosan or carboxymethylcellulose, conditioning medium and surgical extract, in the form of lipoaspirate, in a gas permeant syringe or container in a closed container containing a gas mixture with 0.1% and 5% oxygen, 10% CO₂ and the remainder nitrogen, at 37° C. and ambient pressure, for 3 hours, prior to minimally invasive delivery for volume augmentation in breast reconstruction by injection through 18G canulae into the subcutaneous fat tissue. Nitric oxide (NO) in the gas phase, in concentrations from 10 ppm to 1000 ppm can be added to enhance production of pro-angiogenic factors and particularly VEGFA, or alternatively organic sources of nitrogen oxides such as nitroglycerin (0.1-10 micromolar) can be added to the conditioning medium.

In another embodiment, with the aim of cueing adipogenic differentiation in the stem cell fraction of lipoaspirate, the conditioning medium is commercial high glucose DMEM with either glutamine (1-5 mM) or a peptide source of glutamine such as glutamax (1-10 mM). It is also provisioned with autologous serum at a concentration of 10%, and additionally contains insulin (1 to 10 microgram/mL), and/or transferrin (1-50 mg/L), and/or sodium selenite (1-50 ng/ml) or seleno-L-methionine (50-500 nM), and/or 3-Isobutyl-1-methylxanthine IBMX (0.1 mM to 3 mM), and/or sodium butyrate (0.1 mM to 10 mM), and/or oleic acid (0.05 mM to 5 mM), and/or dexamethasone (0.1-5 micromolar), and/or rosiglitazone (0.1 micromolar to 50 micromolar). Insulin analogs or insulin-like growth factor, other analogs and activators of AMPc, other short chain fatty acids such as sodium propionate or other ketone bodies, other thiazolidinediones such as pioglitazone (0.1 micromolar to 100 micromolar), and/or additionally sulfonylureas such as glimepiride (0.01-1 mM), anti-seizure drugs with adipogenic side effects such as carbamazepine (10 micromolar to 200 micromolar) or glimepiride (10 micromolar to 1 mM) as well as beta-blockers and inhibitors of cyclo-oxygenase-2 (COX-2) such as Celecoxib (0.05 mg/ml to 0.5 mg/mL). This conditioning medium induces adipogenic differentiation. In one embodiment, it is used for incubation of a biological assembly consisting of stromal vascular fraction obtained from lipoaspirate by sedimentation and a final concentration of 1% of collagen I. The assembly is conditioned in a gas permeant syringe or container in a closed container containing a gas consisting of 95% ambient air and 5% CO₂, or a gas mixture containing 20-95% oxygen, 5% CO₂, the remainder being nitrogen for 5 h at 37° C. The conditioned biological extract is injected near the periosteum for volume enhancement in cheeks.

In another embodiment, the surgical isolate in the form of lipoaspirate is split, and part is incubated under partial starvation conditions as part of a biological assembly in a gas permeant syringe or container under hypoxia, while another part, possibly enriched in stem cells, is induced adipogenically in normoxia or hyperoxia, as illustrated above. Upon completion of the conditioning, the fractions are mixed by syringe or container-to-syringe or container connector and repeated transfer from one syringe or container to the other, before injection in soft tissue augmentation or reconstruction in the breast, face, buttock or elsewhere. In this way, pro-regenerative and angiogenic factors Induced in the starved part foster rapid vascularization, while adipgenically primed stem cells reconstruct adipose tissue after establishment of a vascularized implant. In case where conditioning times are significantly different (adipogenic priming or differentiation can also be extended to 1 day to 3 weeks), one or several fractions can be frozen in the presence of cryoprotectants and stored at low temperature (−80° C., −196° C.) as needed.

### f) Regarding the Delivery Fluid

The delivery is fluid delivered within the biological assembly by injection or implantation. The delivery fluid is a biocompatible fluid, and can be identical to conditioning or storage fluid, or different. Delivery fluids can among others be physiological saline, phosphate buffered saline, cell culture medium, plasma, blood and others. The delivery fluid can among others contain anaesthetics, in particular lidocaine, and vasoactive agents such as adrenaline.

### g) Regarding the Biological Extract

The biological extract is conditioned through the use of the devices and methods of this Invention. It may consist of or include tissues, fragmented tissues, liquefied tissues, dissociated tissues, primary cells, cell lines, immortalized cells, extracellular matrix (collagens, laminins, glycosaminoglycanes, chitin, chitosan, hyaluronic acid, matrigel, blood, plasma, serum, biopsies, surgically resected material, induced pluripotent stem cells, transdifferentiated cells, differentiated stem cells, stem cells, cells labelled with contrast-agent, cells labelled with contrast-agents, genetically modified cells, and so forth. Biological extracts can be obtained by mixing different elements or as single isolate. Biological extract can be sourced from the patient destined to receive the biological assembly being prepared according to the invention (autologous source), or from donors different from the patient (heterologous, possibly with immunomatching), from cell banks, from animals (xenologous, possibly humanized or genetically modified to present human antigen determinants).

### h) Regarding the Biomaterial

The biomaterial is biocompatible in-vitro and optionally in-vivo, for example for in-vivo delivery. The biomaterial prevents sedimentation of the biological extract. For this, it can comprise a viscosing agent, a hydrogel, or particulate material, agents permitting to match the specific gravity of the biological extract such as Ficoll or lodixanol, or combinations thereof. In embodiments where it is delivered in a patient, it is injectable. The biomaterial may be porous (100 nm to 1 cm pore size, preferentially between 5 microns and 500 microns) or non-porous, it may be chosen or modified to be cell-adhesive or non-cell-adhesive, or adhesive for specific cell types and non-adhesive for others. It may be chosen to be hydrophilic or hydrophobic, or intermediate, or consist of mixed populations of hydrophobic and hydrophilic regions. It may present enhanced oxygen solubility, and it may be reversibly compressible. A wide variety of chemical composition can be chosen, the biomaterial may for example consist of polymers, polysaccharides, glycosaminoglycanes, cellulose-derivates, carboxymethylcellulose, agarose, peptides, proteins, collagens, laminins, synthetic polymers such as acrylates, polyurethanes, olefins, silicones, fluorinated and perfluorinated polymers, emulsions such as liposomes or perfluorodecalin. The biomaterials may be covalently crosslinked or not. In addition to preventing sedimentation during conditioning, the biomaterial may act as cell support, scaffold, or tissue regeneration template once implanted in-vivo, helping foster ingrowth and vascularization by the host once implanted. It may also influence or modulate the local or systemic immune response, eliciting tolerogenic or inflammatory specific or non-specific responses,

The biomaterial can be of different types, or mixes thereof, including

### Polymer Based Biomaterials:

Hydrogels: extracellular matrix based hydrogel, including collagen, matrigel, laminin; hyaluronic acid based hydrogel, glycosaminoglucane based hydrogels, including chitosan based hydrogel; acrylates; cellulose and its derivatives including carboxymethylcellulose, hydroxyethylcellulose, hydroxypropylcellulose, methylcellulose; agarose; alginate; gelatin; heparin, heparosan, nucleic acids,
Other polymer-based biomaterials: silk-based biomaterial; decellularized plant-based biomaterial; synthetic polymer including polyurethanes, silicones, polyolefins, poly-vinyl-alcohol,
Polymer based porous scaffolds
Ceramics: hydroxyapatite based biomaterial, calcium phosphate,
Extracellular matrix, including collagen, laminin, fibrine, fibronectin
Patient derived materials, including lipoaspirate, bone marrow extract, bone fragments, blood, plasma, fibrin, coagulated blood,

In one embodiment, the biomaterial consists of porous, covalently crosslinked polysaccharide, preferentially covalently crosslinked anionic polysaccharide, and even more preferentially covalently crosslinked hydroxyethylcellulose, carboxymethylcellulose, alginate or hyaluronic acid. The average pore diameter is between 10 micrometers and 10 mm, more preferentially between 20 micrometers and 1 mm, the pore fraction between 10% and 99.9%, more preferentially between 20% and 99%, and most preferentially between 30% and 95%, the total polymer concentration between 5 mg/ml and 100 mg/mL, more preferentially between 10 mg/mL and 80 mg/mL. In one embodiment, the biomaterial consists of a multitude of porous particles, in another, it is monolithic. In one embodiment the particles have a spherical outer shape, in another, their shape is irregular. In one embodiment, biomaterial is reversibly elastically compressible up to 50% volume loss by pore fluid extrusion, more preferentially up to 75%. In one embodiment, the biomaterial has a Young modulus under drained conditions of 10 Pa to 1 MPa, more preferentially 100 Pa to 100 kPa, more preferentially 200 Pa to 20 kPa, most preferentially 500 Pa to 10 kPa.

### i) Additional Method Variations

| A) | B) | C) | D) |
|---|---|---|---|
| Collection | | Collection | |
| Purification | Collection | Mixing | |
| Mixing | Mixing | Conditioning | Collection |
| Conditioning | Conditioning | Decellularization | Addition |
| Medium exchange | Decellularization | Sterilization | Condtitoning |
| | | Storage | |
| Conditioning | Specialized | Specialized | Media |
| | cell type | cell type | exchange |
| Delivery | Delivery | Delivery | Delivery |

Many variations of the basic method of use according to the invention (exemplified in FIG. 2) are possible. The above schematically represented variations A), B), C), and D) show some particular use cases.

For example, variation A) for re-implantation, it may be advantageous to achieve higher purity, functional biological extracts. For this, various purification steps detailed below can be included after primary collection, and also, fluid exchange can be used to remove spent medium with waste products, providing the cells with nutrients for implantation after delivery, yet maintaining secretory and metabolic reprogramming towards a more robust, regenerative or angiogenic phenotype.

In variation B), the primary colonizing cells are used to lay down extracellular matrix for subsequent specialized cell types. Many epithelial cells indeed adhere badly on artificial substrates, but adhere more readily on native matrix. Examples are keratinocytes, hepatocytes, neurons and other specialized cell types. For the application of repairing or engineering injectable functional tissues based on such cell types, a decellularization step can be performed if the primary cells laying down the matrix are not desired. Optionally, in variation C), decellularized products are stored after sterilization and possibly lyophilization.

Variation D) illustrates that the biological extract can be enriched with various exogeneous components. These components can be off-the shelve such as irradiated cell-lines, exosomes, platelets, or other cell products, or also be obtained by clinical isolation.

Optionally, media components can be renewed by addition of through reversible compression cycles; if a porous scaffold is used, continuous perfusion is also possible, for instance with plunger specifically designed with a fluid conduit. To avoid the complexity associated with perfusion, and associated risks of contaminations, it is particularly advantageous to use a reversibly compressible and injectable scaffold (as described e.g. in WO2019068206A1 which regards a soft tissue engineering material comprising a multitude of particles consisting of three-dimensionally warped and branched sheets), that allows for evacuation of spent medium via gentle, but efficient compression, and subsequent admixing of new media. In this way, sequential differentiation protocols can be carried out in-situ, or additional steps such as decellularization and even reseeding of decellularized material with new cell types can be carried out.

The biological extract to be mixed with the biomaterial can also include various biological fractions and pre-processing steps. For instance, various techniques can be used to isolate desired elements such as the stromal-vascular fraction or platelets, or in fact any target cells from the original isolate. These enrichment techniques are well known in the art and consist in techniques such as centrifugation, size-based filtering, but also more specific techniques such as magnetic enrichment, fluorescence activated cell sorting and others after specific or differential labelling of target and non-target cell populations by means of antibodies, nucleic acid probes, and the like. Cell adhesion can also be used as a selection criterion, particularly when applying flow through a porous medium; cell survival can also be a selection criterion, particularly when using lysing buffers for certain cell types such as red blood cells.

In one embodiment, the conditioning syringe or container is used without the presence of a biomaterial and/or without the presence of a conditioning fluid. In one example, the conditioning syringe or container is used to store a first biological extract, conditioning it and then to extract a second biological extract to prepare a mix of two biological extracts. On one embodiment, the first biological extract is bone marrow aspirate harvested from the patient and the second biological extract is lipo-aspirated material.

In one embodiment, a decellularization process is carried out after the collection of the biological extract in the conditioning syringe or container, after the conditioning or without conditioning step. In a further embodiment, after decellularization, a second biological extract is added.

The temperature of incubation is often chosen near body temperature, but there are situations where a lower temperature may be more adequate, be it for maintaining viability or for readjustment of the circadian stage of the isolate, or also for storage.

It is obviously also possible to combine lipoaspirate, plasma or other biological extracts, among themselves, or with cell culture and expansion products such as amplified cells (keratinocytes, fibroblasts, immune cells, stem cells, neural stem cells, endothelial cells hepatocytoes, and many more), and also non-living components (agents of genetic modification such as viruses, transfection agents, nucleic acids; exosomes; conditioned medium; extracellular matrix and others). There is also wide variation possible regarding the viscous medium holding cells in place. Cell-adhesion to components of the medium is not necessary for the basic functionality of the device. To prevent sedimentation and maintain homogeneous cell distribution, viscosing agents such as polyvinylalcohol, or uncrosslinked acrylates such as polyacrylamide, polyhydroxyethylacrylamide, polyacrylic acid sodium salt, and other acrylates, and also co-polymers of various acrylates can be used. To enhance viscosity, phase transition materials can also be employed, for instance agarose, which gels upon temperature lowering, or PNIPAAM of methylcellulose which on the contrary gel upon temperature elevation. Shear thinning hydrogels, either homogeneous such as carboxymethylcellulose or hyaluronic acid and others can be advantageously used as they will increase in apparent viscosity after the mixing step(s).

Crosslinked, and partially crosslinked hydrogels can also be used, in a particularly preferred embodiment as a dense suspension of interlocking porous particles of crosslinked hydrogels. As discussed, a particularly advantageous combination is offered by porous, interlocking particles that are additionally reversibly elastically compressible. Such particles are easily stored in a partially hydrated state, that is with a substantial swelling capacity. This simultaneously, and surprisingly synergistically provides the capacity to aspirate elements of the biological extract, in fixed homogeneous spatial relation ensuring optimal conditioning. If required, the interlocking and ensuing stability permits facile, rapid and nearly complete medium exchange by transient compression while protecting cells from evacuation from the biomplant. At the same, the particles are able to transiently glide among themselves, and transiently be compressed, permitting fluidification for minimally invasive delivery while protecting the biological extract lodged in the pore space. The porous structure (with pore diameters preferentially in the range between 10 micrometers and 5 mm, more preferentially 20 micrometers and 1 mm) in-vivo fosters regeneration and vascularization, while during conditioning, it offers unprecedented surface area for storage of the secretome. Different types of particles can be mixed, including ones with different capacity for storing different parts of the secretome, and different Young moduli, to foster locally different tissue differentiation and thus the generation of locally patterned tissues.

To further facilitate oxygen diffusion, emulsion of enhancer of oxygen solubility can also be used, such an emulsion of perfluorodecalin or other perfluorinated liquids imprisoned in biomaterial particles or bulk material.

In one embodiment, the bioimplant is compressible and can be delivered as single, cohesive piece for minimally invasive or open surgery. In one embodiment, the bioimplant can be sutured to existing tissues. In another embodiment, the bioimplant has an anisotropic pore structure, enabling preferential growth of nerve, muscle, vascular and lymphatic elements along the main direction. The pore anisotropy index, defined as the ratio between long and short pore diameter as measured by maximal ellipses on confocal images, is preferentially between 1 and 100, more preferentially between 2 and 50.

The biological extract becomes physically upon mixing entrapped or entangled with the porous material and may also optionally develop biological adhesion. The biological assembly can then be placed in a perfusion syringe or container, in which it can be perfused for as long as the perfusion rate remains below the critical threshold for inducing shear yielding. This enables precise control over the medium condition, either be continuous perfusion or by pre-defined exchange protocols. After a suitable incubation time at suitable temperatures, the syringe or container is disconnected from the perfusion and is ready for use in minimally invasive implantation. Means of achieving enhance regenerative potential can be varied, they can include among others low oxygen concentrations, enhanced acidity, low nutrient availability, heat shock, mitochondrial decoupling agents, ketone bodies such as acetone or hydroxybutyric acid, irradiation, immune and pain mediators such as histamine or substance P.

An important element of the present invention is the use of gas permeant syringe or container. Indeed, most cell types do not support extended duration of deep or complete hypoxia, nor deep acidification upon accumulation of carbon dioxide and anaerobic fermentation products such as lactic acid. Controlled moderate hypoxia on the other hand may be beneficial in gearing cells towards the production of a favorable metabolic and secretory profile. Indeed, controlled moderate hypoxia can be used to induce a proangiogenic and proregenerative secretory profile.

Membranes and other solutions permitting rapid gas exchange are readily available, and can be welded or glued to cylindrical perforated backing shape for assembly of a device as shown in FIG. 6. Gas permeant membranes exist and are known in the art such as silicones and perfluorinated polymers exist and can be used to mold gas permeant syringe or containers for the present invention. For instance, WO2005035728 discusses a number of options, among others robust silicones, and indeed, a 0.3 mm thick silicone rubber (polyalkylsiloxane) wall has a gas permeability of about 0.15 mL O₂*m²*day⁻¹*Pa⁻¹. The syringe or container backing can be assembled by fabrication techniques known in the art, for instance, 3D printing can be used, or classical mechanical manufacture of a perforated shape from stainless steel. At a larger scale, injection molding of thermaplasts such as polypropylene, cycloolefin copolymers and other materials well known in the art of syringe or container production can be used. Advantageously, gas exchange through thin hydrophobic membranes enables direct gas-liquid contact while containing the liquid due to capillary effects. For example, hydrophobic membranes (for instance, from PTFE) with 0.22 μm or better 100 nm pores can support many atmospheres of pressure before yielding to syringe or container pressure, while permitting rapid gas exchange. Such membranes are commercially available and can be welded or glued into cylindrical form, and be supported by a suitable mechanical structure. They permit to maintain sterility within the syringe or container.

The conditioning fluid can be used to decrease contamination probability. For this, a disinfectant with antibacterial activity but compatible with mammalian cell survival can be used, for example iodine-based disinfectants such as betadine (e.g. 0.1 mg/ml to 11 mg/mL, more preferentially 0.3 mg/ml to 3 mg/mL of iodine, for a duration of 1 minute to 30 minutes, more preferentially 5 minutes to 20 minutes). In some embodiments, immunomodulatory action of the conditioning is achieved. Tolerogenic action can be achieved by glucocorticoids, but also gamma-irradiation or other treatment suitable to achieve a substantial fraction of apoptotic cells (preferentially larger than 5%, more preferentially larger than 10%, most preferentially larger than 25%). Immune activation on the other hand can be achieved by check-point blockade antibodies and agents known in the art (anti-PD1, anti-CTLA4, and others), and/or by adjuvants (poly-IC, aluminum salts, CpG oligonucleotides, and others).

After incubation for a defined time, at a defined temperature (preferentially, between 20° C. and 45°, more preferentially between 30° and 40″, and even more preferentially between 35° C. and 38° C.) and controlled gas atmosphere, the cellular or biological isolates are suitably conditioned and can be injected for autologous or possibly heterologous or xenologous transplantation. The hydrogel or scaffold needs to be suitable for accepting the cells upon mixing, yet avoid sedimentation to maintain homogeneous conditions, and the syringe or container must be sufficiently gas permeant to allow for external control over gas exchange.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Biomaterials preferentially exhibit Young moduli or shear moduli (G′) in the range of the target tissues, i.e. between 100 Pa and 100 kPa for soft tissues, more preferentially between 200 Pa and 20 kPa and most preferentially between 500 Pa and 10 kPa. Young moduli for polymer-based materials may be adjusted by means known in the art, such as choice of base material, choice of pore fraction, choice of crosslinker and choice of crosslinking density, if crosslinked materials are used.

1-56. (canceled)

57. A sterile container, wherein:
(i) the container has an oxygen and/or carbon dioxide permeability of at least 0.1 mL/Pa/m²/day;
(ii) the container has at least one fluidic port permitting sterile connection to other containers and/or tubes;
(iii) the container contains a porous, biocompatible polymer-based biomaterial with at least 30% porosity;
(iv) the porous biomaterial has a pore size of between 20 micrometers and 2 mm;
(v) the porous biomaterial has a Young's modulus of 50 Pa to 50 kPa; and
(vi) the porous biomaterial is reversibly compressible by at least a factor of 2.

58. The container according to claim 57, wherein the porous biomaterial has a swelling capacity of at least 10%.

59. The container according to claim 57, wherein the porous biomaterial has a pore size of between 50 micrometers and 1 mm.

60. The container according to claim 57, wherein the porous biomaterial has a Young's modulus of 200 Pa to 20 kPa.

61. The container according to claim 57, wherein the container is a gas-permeant syringe.

62. The container according to claim 57, wherein the porous polymer-based biomaterial comprises any one or more of:
(i) polysaccharides;
(ii) patient-derived materials; and
(iii) extracellular matrix.

63. The container according to claim 57, wherein the porous polymer-based biomaterial comprises covalently crosslinked polysaccharides.

64. The container according to claim 57, wherein the porous polymer-based biomaterial comprises one or more selected from the group consisting of:
extracellular matrix-based hydrogel;
collagen;
matrigel;
laminin;
elastin;
polysaccharide-based hydrogels;
hyaluronic acid-based hydrogel;
glycosaminoglucane based hydrogels;
chitosan-based hydrogel;
cellulose and its derivatives;
carboxymethylcellulose;
hydroxyethylcellulose;
hydroxypropylcellulose;
methylcellulose;
agarose;
alginate;
gelatin;
heparin,
heparosan,
nucleic acids;
acrylates;
silk-based biomaterials;
decellularized plant-based biomaterials;
synthetic polymers;
polyurethanes;
silicones;
polyolefins; and
polyvinyl alcohol.

65. The container according to claim 57, wherein the porous biomaterial has a viscosity of at least 0.02 Pa·s.

66. The container according to claim 57, wherein the container comprises at least one inlet port for admitting a cell suspension or biological extract for forming a bioimplant by conditioning the cell suspension or biological extract in the container, and an outlet port for delivery of the bioimplant from the container.

67. The container according to claim 57, wherein the porous biomaterial has a partition coefficient for a target growth factor comprised between 1 and 1,000,000.

68. The container according to claim 67, wherein the target growth factor is one or more selected from the group consisting of:
a growth factor;
a paracrine factor;
an antibody;
an hormone;
a lipid mediator;
a prostaglandin;
an inhibitor;
a soluble receptor;
an exosome;
a lipoprotein;
a protein;
a peptide;
a lipid;
an amine;
a neurotransmitter;
a decarboxylated amino acid;
an RNA;
DNA;
a cytokine;
a chemokine;
a neuropeptide;
a carbohydrate;
an extracellular matrix protein;
a metabolite;
an enzyme;
a marker;
a radiotracer;
an immunomodulator; and
a gas.

69. The container according to claim 67, wherein the container is a syringe,
wherein the syringe has a barrel comprising an amount of polymer-based biomaterials and one or both of:
(i) patient derived materials; and
(ii) an extracellular matrix having a viscosity above 0.02 Poise; and
wherein that the wall of the barrel exhibits an overall oxygen permeability of at least 0.1 mL/Pa/m²/day as measured under standard pressure and temperature.

70. The container according to claim 69, wherein the polymer-based biomaterials are one or more selected from the group consisting of:
hydrogels;
extracellular matrix-based hydrogel;
collagen;
matrigel;
laminin;
hyaluronic acid-based hydrogel;
glycosaminoglucane based hydrogels;
chitosan-based hydrogel;
acrylates;
cellulose and its derivatives;
carboxymethylcellulose;
hydroxyethylcellulose;
hydroxypropylcellulose;
methylcellulose;
agarose;
alginate;
gelatin;
heparin;
heparosan
nucleic acids;
silk-based biomaterial;
decellularized plant-based biomaterial;
synthetic polymer;
polyurethanes;
silicones;
polyolefins;
poly-vinyl-alcohol;
polymer based porous scaffolds;
ceramics;
hydroxyapatite-based biomaterials; and
calcium phosphate.

71. The container according to claim 69, wherein the overall oxygen permeability is at least 1 mL/Pa/m²/day.

72. The container according to claim 70, wherein the viscosity of the hydrogel is above 0.1 Poise.

73. The container according to claim 70, wherein the syringe comprises an amount of hydrogel corresponding to 0.01%-99.00% of the syringe volume such that the syringe is not entirely filled.

74. The container according to claim 70, wherein the syringe further comprises an amount of conditioning fluid, said conditioning fluid comprising:
(i) 0.1 mM to 3 mM of glucose or 2-deoxy-D-glucose; or
(ii) any one or more of dimethyl sulfoxide, glycerol, mannitol, trehalose or other small molecules suitable for cryopreservation, cryoprotectants with macromolecular constituents, blood plasma and polyvinyl alcohol.

75. The container according to claim 74, wherein the polymer-based biomaterial is a hydrogel reversibly compressible by at least a factor of 3.

76. An assembly comprising a container according to claim 57 and a closable container, wherein:
(i) the closable container is configured to sealingly house the container according to claim 57 therein; and
(ii) the closable container comprises a controlled gas composition comprising an amount of oxygen.

77. The assembly according to claim 76, wherein the gas composition comprises between 2 and 15% of carbon dioxide.
